(19) United States
(12) Patent Application Publication
(10) Pub. No.: US 2021/0313072 A1
(43) Pub. Date: Oct. 7, 2021

(54) DYNAMICALLY EVALUATING HEALTH CARE RISK

(71) Applicant: b.well Connected Health, Inc., Eldersburg, MD (US)

(72) Inventors: Imran Iqbal Qureshi, Walnut Creek, CA (US); Sean Paul Hegarty, San Francisco, CA (US)

(21) Appl. No.: 17/351,095

(22) Filed: Jun. 17, 2021

Related U.S. Application Data
(63) Continuation-in-part of application No. 15/800,772, filed on Nov. 1, 2017, now Pat. No. 11,004,547.
(60) Provisional application No. 62/416,106, filed on Nov. 1, 2016.

(51) Int. Cl. G16H 50/70; G16H 50/30; G16H 10/60; G06Q 40/08; G06Q 50/00; G06F 16/901; G06F 16/23; G06N 5/02

(52) U.S. Cl. CPC G16H 50/70; G16H 50/30; G16H 10/60; G06Q 40/08; G16H 40/20; G06F 16/9024; G06F 16/2379; G06N 5/02; G06Q 50/01

(57) ABSTRACT

A health risk evaluation system may manage, for a set of users, a corresponding set of digital healthcare profiles, where each digital healthcare profile includes a healthcare decision tree; determine, for a first user, a set of possible actions related to improving health of the first user; determine, for each of the set of possible actions, an impact of the recommendation; determine, from the set of possible actions, a first action based on an impact of the first action; and provide, to the first user, the first action from the set of possible actions.

### CROSS-REFERENCE TO OTHER PATENT APPLICATIONS

This application is a continuation-in-part of U.S. patent application Ser. No. 15/800,772, which has been incorporated by reference herein in its entirety.

### BACKGROUND

Electronic healthcare records (EHR) are generated and stored according to various company-specific, product-specific, or standardized data models, such as HL7 and FHIR. But healthcare software applications may use variants of standard data models or customized data models that are not openly available. Moreover, EHR health data is often fragmented, but still growing more so by the day. Consequently, stakeholders (e.g., patients, insurers, care providers, employers) do not have access to comprehensive datasets that needed to control health outcomes, and consumers historically have not had any access to their own health data. Additionally, as user devices and products moving into a digital age where consumers can access fitness, nutrition, diagnosis and have medical encounters all online through a growing number of platforms and providers, there is a growing need for data to be aggregated and standardized across disparate data sources.

The usage of this aggregated and standardized health care data is in its infancy as well. Determining useful information and actionable recommendations for a user related to their health care can be incredibly difficult, given the increasing amount of data from disparate data sources about individuals and their healthcare data.

### BRIEF DESCRIPTION OF THE DRAWINGS

The following detailed description references the drawings, wherein:

FIG. 1 is a block diagram depicting an example environment in which various examples may be implemented as a dynamic health care risk evaluation system.

FIG. 2 is a block diagram depicting an example environment in which various examples may be implemented as a dynamic health care risk evaluation system.

FIG. 3 is a diagram depicting an example set of correlated users.

FIG. 4 is a block diagram depicting an example machine-readable storage medium comprising instructions executable by a processor for dynamically evaluating health care risk.

FIG. 5 is a flow diagram depicting an example method for dynamically evaluating health care risk.

### DETAILED DESCRIPTION

The following detailed description refers to the accompanying drawings. Wherever possible, the same reference numbers are used in the drawings and the following description to refer to the same or similar parts. It is to be expressly understood, however, that the drawings are for the purpose of illustration and description only. While several examples are described in this document, modifications, adaptations, and other implementations are possible. Accordingly, the following detailed description does not limit the disclosed examples. Instead, the proper scope of the disclosed examples may be defined by the appended claims.

Electronic healthcare records (EHR) are generated and stored according to various company-specific, product-specific, or standardized data models, such as HL7 and FHIR. But healthcare software applications may use variants of standard data models or customized data models that are not openly available. Moreover, EHR health data is often fragmented, but still growing more so by the day. Consequently, stakeholders (e.g., patients, insurers, care providers, employers) do not have access to comprehensive datasets that needed to control health outcomes, and consumers historically have not had any access to their own health data. Additionally, as user devices and products moving into a digital age where consumers can access fitness, nutrition, diagnosis and have medical encounters all online through a growing number of platforms and providers, there is a growing need for data to be aggregated and standardized across disparate data sources.

The usage of this aggregated and standardized health care data is in its infancy as well. Determining useful information and actionable recommendations for a user related to their health care can be incredibly difficult, given the increasing amount of data from disparate data sources about individuals and their healthcare data.

Examples disclosed herein provide technical solutions to these technical challenges by dynamically evaluating health care risk in an automated way that enables health care action recommendations for a user based on their healthcare profile and healthcare decision tree, and based on the profiles and healthcare decision trees of other users that have social relationships to that user. By determining cohorts of users that are similar to the user, a health care risk evaluation system may determine health care actions that may be better suited for the user based on the likelihood that a user will actually engage with or execute on recommended actions because of similar lifestyles. Further, the health care risk evaluation system may determine an impact of each of the determined health care actions and provide information about an action to the user based on their impact. As such, these technical solutions correlate healthcare decision trees between users to understand historical success related to similar people and applies that historical success to the health care action recommendations. The solutions described herein enable an improved and effective analysis and recommendation of health care actions from complicated, large data sets related to users, health care, pharmacy, insurance, providers, and other medical data related to managing user health care. Further, the technical solutions disclosed herein also enable optimization of the health care recommendations for a user in a myriad of ways.

Some examples disclosed herein to dynamically evaluate health care risk include managing, for a set of users, a corresponding set of digital healthcare profiles; determining, for a first user of the set of users, a set of possible actions related to improving health of the first user; determining, for each of the set of possible actions, an impact of the recommendation; determining, from the set of possible recommendations, a first action based on an impact of the first action; and providing, to the first user, information related to the first action from the set of possible actions

Some of the examples disclosed herein to dynamically evaluate health care risk include a system comprising a physical processor implementing machine readable instructions that cause the system to: manage, for a set of users, a corresponding set of digital healthcare profiles; determine, for a first user of the set of users, a set of possible actions related to improving health of the first user; determine, for each of the set of possible actions, an impact of the action; determine, from the set of possible actions, a first action based on an impact of the first action; and provide, to the first user, the first action from the set of possible actions.

Some examples disclosed herein to dynamically evaluate health care risk include a non-transitory machine-readable storage medium comprising instructions executable by a physical processor of a computing device for dynamically evaluating health care risk, the machine-readable storage medium comprising: instructions to manage, for a set of users, a corresponding set of digital healthcare profiles; instructions to determine, for a first user of the set of users, a set of possible actions related to improving health of the first user; instructions to determine, for each of the set of possible actions, an impact of the recommendation; instructions to determine, from the set of possible actions, a first action based on an impact of the first action; and instructions to provide, to the first user, the first action from the set of possible actions.

The terminology used herein is for the purpose of describing particular embodiments only and is not intended to be limiting. As used herein, the singular forms “a,” “an,” and “the” are intended to include the plural forms as well, unless the context clearly indicates otherwise. The term “plurality,” as used herein, is defined as two or more than two. The term “another,” as used herein, is defined as at least a second or more. The term “coupled,” as used herein, is defined as connected, whether directly without any intervening elements or indirectly with at least one intervening elements, unless otherwise indicated. Two elements can be coupled mechanically, electrically, or communicatively linked through a communication channel, pathway, network, or system. The term “and/or” as used herein refers to and encompasses any and all possible combinations of one or more of the associated listed items. It will also be understood that, although the terms first, second, third, etc. may be used herein to describe various elements, these elements should not be limited by these terms, as these terms are only used to distinguish one element from another unless stated otherwise or the context indicates otherwise. As used herein, the term “includes” means includes but not limited to, the term “including” means including but not limited to. The term “based on” means based at least in part on.

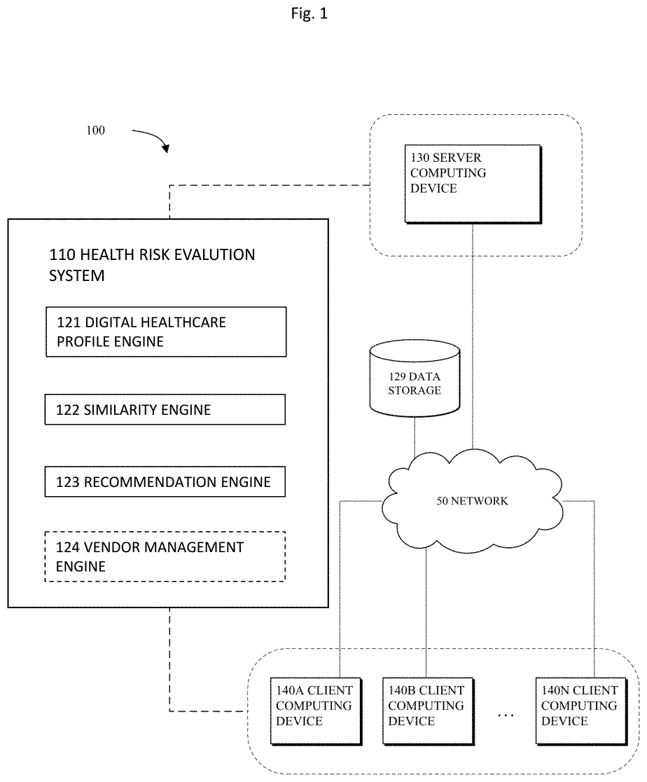

FIG. 1 is an example environment 100 in which various examples may be implemented as a health risk evaluation system 110. In some examples, environment 100 may include various components including server computing device 130 and mobile devices 140 (illustrated as 140A, 140B, . . . , 140N). Each client computing device 140A, 140B, . . . 140N may communicate requests to and/or receive responses from server computing device 130. Server computing device 130 may receive and/or respond to requests from mobile devices 140. Mobile devices 140 may be any type of mobile computing device capable of sending and/or receiving data to server computing device 130. For example, mobile devices 140 may include a laptop computing device, an all-in-one computing device, a thin client, a workstation, a tablet computing device, a mobile phone, an electronic book reader, a network-enabled appliance such as a “Smart” speaker, a network-connected radio, a software defined radio, wideband tuner, and/or other electronic device suitable for collecting data and transmitting that data to the server computing device 130. While server computing device 130 is depicted as a single computing device, server computing device 130 may include any number of integrated or distributed computing devices serving at least one software application for consumption by mobile devices 140. Data store 129 can be any non-transitory machine-readable storage. In some examples, data store 129 can comprise a Solid State Drive (SSD), Hard Disk Drive (HDD), a database, a networked database storage system, a cloud storage, and/or other type of data store that stores information related to health risk evaluation system 110.

The various components (e.g., components 129, 130, and/or 140) depicted in FIG. 1 may be coupled to at least one other component via a network 50. Network 50 may comprise any infrastructure or combination of infrastructures that enable electronic communication between the components. For example, network 50 may include at least one of the Internet, an intranet, a PAN (Personal Area Network), a LAN (Local Area Network), a WAN (Wide Area Network), a SAN (Storage Area Network), a MAN (Metropolitan Area Network), a wireless network, a cellular communications network, a Public Switched Telephone Network, and/or another network.

According to various implementations, health risk evaluation system 110 and the various components described herein may be implemented in hardware and/or a combination of hardware and programming that configures hardware. Furthermore, in FIG. 1 and other Figures described herein, different numbers of components or entities than depicted may be used.

Health risk evaluation system 110 may comprise a digital healthcare profile engine 121, a similarity engine 122, a recommendation engine 123, and/or other engines. The term “engine”, as used herein, refers to a combination of hardware and programming that performs a designated function. As is illustrated respect to FIG. 4, the hardware of each engine, for example, may include one or both of a processor and a machine-readable storage medium, while the programming is instructions or code stored on the machine-readable storage medium and executable by the processor to perform the designated function.

Digital healthcare profile engine 121 may manage medical data related to a set of users. The medical data may comprise patient data, provider data, insurer data, lab data, wearable device data, pharmacy data, actuarial data, and/or other data related to health of a user. The medical data may be obtained from numerous sources, with similar or different formats of data. The digital healthcare profile engine 121 may standardize and create a healthcare digital profile (e.g., a digital health record) for each user of the health risk evaluation system 110, as described in U.S. patent application Ser. No. 15/800,772. For example, the digital healthcare profile engine 121 may anonymize and aggregate the digital health care records of each user to create the healthcare digital profile. In some of these examples, personally identifiable information may be kept separate from the healthcare digital profile. Given that, the data in the healthcare digital profile is anonymized, and any calculations, aggregations, statistics, and/or other analysis may be performed on the anonymized healthcare digital profile. In some examples, the digital healthcare profile engine 121 may create and/or manage a digital health care profile in a manner similar to or the same as described in co-pending U.S. patent application Ser. No. ______, filed on the same day as this current application, and incorporated by reference herein in its entirety.

The digital healthcare profile engine 121 may generate and manage a healthcare decision tree for each user as well, where a healthcare decision tree may be part of the healthcare digital profile for each user. The healthcare decision tree for a user may comprise information about a set of actions. An action may comprise, for example, a recommendation, a doctor's visit, prescription, refill, exercise, weight change, activity, and/or other act. The information about an action may comprise, for example, a date of the action, time of the action, title for the action, description of the action, indicator of whether the action was performed or not, provider(s) associated with the action, insurer(s) associated with the action, lab result(s) associated with the action, prescription(s) associated with the action, other user(s) associated with the action, and/or other data related to the action. The healthcare decision tree for a user may represent a chronological order of actions based on a date/time of each action.

In some examples, information about an action may also comprise a score associated with the action. The score may indicate: a state of health associated with the action, a health care cost associated with the action, a state of health associated with the healthcare decision tree up until that action, a health care cost associated with the health care decision tree up until that action, a priority related to the performance of the action, likelihood of engagement in the action, motivation to perform the action, health history associated with the action, treatment risk associated with the action, mortality rate associated with performing the action, mortality rate associated with not performing the action, life expectancy associated with performing the action, life expectancy associated with not performing the action, patient satisfaction associated with performing the action, patient satisfaction associated with not performing the action, total cost associated with performing the action, total cost associated with not performing the action, user preference associated with performing the action, health care provider preference associated with performing the action, and/or other factors related to the action. In some examples, a score associated with an action may be determined based on calculating and/or aggregating multiple level of scores. In these examples, the factors related to mortality rate, life expectancy, total cost, and/or other factors may be treated as a set of sub-scores that can be aggregated or otherwise used to determine an overall score for the health care cost associated with the action.

In some examples, a score or sub-score may be determined based on machine learning by the system, user interaction with actions along the user's healthcare decision tree, user preferences, user demographics, average scores of actions or healthcare decision trees users with similar healthcare decision trees, hashing, statistical measures, regressions, classifiers, clusterings, Bayesian methods, any combination thereof, and/or other mathematical calculations. In some examples, the score for an action, and/or each sub-score associated with the action, may be weighted based on machine learning by the system, user interaction with actions along the user's healthcare decision tree, user preferences, user demographics, average scores of actions or healthcare decision trees users with similar healthcare decision trees, hashing, statistical measures, regressions, classifiers, clusterings, Bayesian methods, types of relationships between the users, any combination thereof, and/or other mathematical calculations. Scores and/or weights may be updated regularly at a scheduled time and/or updated responsive to changes in a healthcare decision tree of the user or other user in the system, other change in data in the system, and/or other factor.

In some examples, the healthcare decision tree may also comprise relationships between actions. For example, a relationship between a set of actions may comprise actions linked to each other based on one action resulting in another action. In one example, a set of linked actions may include a doctor visit, a prescription ordered by the doctor, a test ordered by the doctor, lab result(s) from the test, and/or other actions linked to the doctor visit. In another example, a set of linked actions may comprise actions associated with a condition that the user has, including recommended exercise actions as part of a treatment for the condition, a set of weight recordings obtained at specific time intervals, a set of symptom data related to the condition that are obtained at specific time intervals, and/or other data related to the condition and recommended treatment. In some examples, the actions between the doctor visit and the condition may be linked as well. The links may comprise different types of links that indicate a type of relationship of the link, such as symptoms, treatments, time-based, activity-based, recommendation-based, and/or other type of relationship. In some examples, there may be multiple types of links between actions.

As such, the healthcare decision tree may include information about the healthcare journey of the user, with actions and dates/times of the actions, related to the health of the user and related to other users based on different types of links. In some examples, the relationships between actions may also be scored, with sets of scores/sub-scores, in a manner the same as or similar to scoring an individual action described above. In some examples, the healthcare decision tree may be scored as well, with sets of scores/sub-scores, in a manner the same as or similar to scoring an individual action or relationships between actions described above.

The digital healthcare profile engine 121 may update the healthcare decision tree for a user continuously, as actions are recommended, performed, removed, and/or otherwise changed for the user. For example, the digital healthcare profile engine 121 may update the healthcare decision tree of a user based on received data about a doctor's visit, prescription filling, exercise undertaken, weight change, activity level, performance of a recommended action, and/or other change or addition of an action by the user. In some examples, the digital healthcare profile engine 121 may update the healthcare decision tree of a user based on receipt of data from one or multiple numerous sources of data from which medical data is obtained by the digital healthcare profile engine 121.

The similarity engine 122 may determine, for a first user of the set of users, a first subset of users with a first set of similar digital healthcare profiles. The similarity engine 122 may determine the first subset of users (among the set of users) based on a similarity between the healthcare decision trees of the first subset of users and a first healthcare decision tree of the first user. In some examples, the similarity engine 122 may normalize each of the healthcare decision trees of the users of the health risk evaluation system 110, and may determine a similarity of the healthcare decision trees with the first user based on one or multiple metrics related to the normalized healthcare decision trees.

In some examples, the similarity engine 122 may determine similarity of a healthcare decision tree by hashing, statistical measures, regressions, classifiers, clusterings, Bayesian methods, any combination thereof, and/or other mathematical calculations. In some examples, the similarity engine 122 may use data science, machine learning, artificial intelligence, and/or other mathematical calculations to determine a similarity of the healthcare decision trees. For example, the similarity engine 122 may consider actions of the healthcare decision tree, attributes of the digital healthcare profile, metadata related to the healthcare decision tree, metadata related to the profile, and/or other data in the system 110 to determine a similarity of the healthcare decision trees.

In some of these examples, the similarity engine 122 may determine how many actions are similar between the healthcare decision trees, and may determine how many differ. The differing actions may be considered inflection points, and the similarity engine 122 may determine whether the one or multiple inflection points between two healthcare decision trees are different enough that they would not be considered similar. In some of these examples, the similarity engine 122 may determine similarity based on statistically significant closeness of the healthcare decision trees. In some of these examples, the similarity engine 122 may determine similarity based on a subset of healthcare decision trees within a standard deviation, a predetermined statistically significant amount, and/or other similarity metric. The similarity engine 122 may use a predetermined threshold for each of the one or multiple metrics used to determine whether a healthcare decision tree is similar to the first healthcare decision tree of the first user.

In some examples, the similarity engine 122 may determine multiple sets of similar healthcare decision trees to the first healthcare decision tree of the first user. The similarity engine 122 may determine each set of similar healthcare decision trees based on one or multiple actions in the user's healthcare decision tree, one or multiple sets of linked actions, social relationships between users in the health risk evaluation system 110, and/or based on other factors.

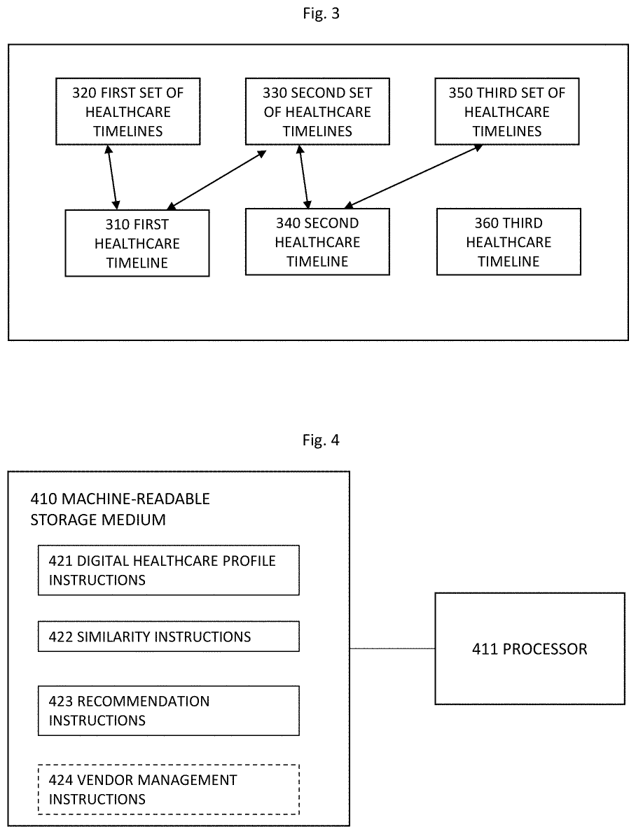

For example, as shown in FIG. 3A, a first user healthcare decision tree 310 may be correlated to a first set of healthcare decision trees 320 and a second set of healthcare decision trees 330. As depicted in the example, a second user healthcare decision tree 340 correlated with the second set of healthcare decision trees 330 may also be correlated with a third set of healthcare decision trees 350, which the first user healthcare decision tree is not correlated with. In yet another example, a third healthcare decision tree 360 may not be correlated with the first, second, or third sets of healthcare decision trees 320, 330, 350. In some examples, the system 110 may determine that a second healthcare decision tree 340 of a second user may be included in both the first set of healthcare decision trees 320 and the second set of healthcare decision trees 330. The number and types of correlations between the user healthcare decision trees of the system 110 are not limited to the examples described herein.

In some of these examples, the first set of healthcare decision trees 320 may comprise a set of healthcare decision trees determined above with respect to the metrics and actions described above, and the second set of healthcare decision trees 330 may correspond to a second set of healthcare decision trees associated with a second set of users who each individually have a social relationship to the first user. In these examples, the health risk evaluation system 110 may store information related to social relationships between users (e.g., family members, friends, co-employees, peers, members of a same sports group/gym, persons in a same club or part of a same membership, neighbors, and/or other types of relationships). The system 110 may also determine secondary, tertiary, and nth-level social relationships (e.g., friends of friends, persons who are members of a same club as a co-employee, relatives of your children's friends, etc.) based on the stored data related to relationships between users. In some examples, the digital healthcare profile engine 121 may determine a set of healthcare decision trees based on relationships to the user in a manner similar to or the same as described in co-pending U.S. patent application Ser. No. ______, filed on the same day as this current application, and incorporated by reference herein in its entirety.

In some examples, the system 110 may determine that a social relationship may exist between the first user and a second user based on data stored for each user (e.g., medical records, legal records, proximity of the activities associated with the first and second user, and/or based on other data of the first and second user). In some examples, the system 110 may request confirmation of the determined relationship between the first user and the second user. In some examples, the system 110 may receive data from the first user related to relationships between the first user and other users of the system 110.

In some examples, the health risk evaluation system 110 may store information related to each relationship of the first user, including, for example, a unique identifier of the second user, a name of the second user, a date at which the relationship started, a date at which the relationship was confirmed, a type of the relationship (e.g., immediate family, secondary family, friend, co-worker, and/or other relationship type), a relationship level (e.g., first level—personally known; secondary level; tertiary level, and/or other nth level of relationship), an end date of the relationship, and/or other information related to the relationship between the first user and the second user.

In some examples, the system 110 may also store a set of relationship type weights, where each weight is associated with a type of relationship. In some of these examples, a higher weight may be associated with a “closer” social relationship. For example, a relationship type indicating a family member may have a higher weight than a relationship type indicating a co-worker. In some examples, the system 110 may store a set of relationship level weights, where each weight is associated with a relationship level. In some of these examples, a higher weight may be associated with a “closer” social relationship. For example, a relationship level indicating a first level relationship (e.g., parent-child relationship) may have a higher weight than a relationship level indicating a secondary level relationship (e.g., aunt-nephew relationship). In some examples, the system 110 may determine weight for the social relationship between the first user and a second user based on the relationship type weight, the relationship level weight, or some calculation or determination based on both the relationship type weight and the relationships level weight.

Returning to FIG. 1, in some examples, the similarity engine 122 may update the similar healthcare decision trees to the first healthcare decision tree of the first user based on continuously (or at predetermined intervals) running the calculations to determine the similar healthcare decision trees. This updating could be based on updated data to existing healthcare decision trees, new healthcare decision trees being added to the system 110, and/or no changes in data at all (e.g., updates to the calculations themselves to better optimize selection of similar healthcare decision trees).

In some examples, upon a healthcare decision tree being updated (e.g., with a new healthcare action, with updated data related to an existing healthcare action, a new social relationship between the first user and the user associated with the healthcare decision tree, etc.), the similarity engine 122 may determine that the updated healthcare decision tree that had been considered similar has deviated from the set of similar healthcare decision trees based on the one or multiple metrics applied to the updated healthcare decision tree. For example, the similarity engine 122 may determine that the updated healthcare decision tree is statistically significantly different from the set of healthcare decision trees similar to the first healthcare decision tree, may determine that the updated healthcare decision tree is statistically closer to a different set of healthcare decision trees, and/or may otherwise determine that the updated healthcare decision tree deviates from the set of healthcare decision trees similar to the first healthcare decision tree.

Recommendation engine 123 may determine, based on the correlation between the first healthcare decision tree and the set of similar healthcare decision trees, a first recommended action for the first user. In some examples, recommendation engine 123 may determine the first recommended action for the first user from a set of recommended actions determined by the engine 123.

The recommendation engine 123 may determine a set of existing actions in the first user's healthcare decision tree from which to provide recommendations. For example, the recommendation engine 123 may select one or multiple actions to provide recommendations about, based on an action or set of related actions being stored or updated within a predetermined time period, an action or set of related actions having a score or sub-score related to a metric above a predetermined threshold, a user- or provider-initiated request for recommended actions, an action or set of related actions triggering an determination that a recommendation is required to be provided, a treatment plan related to the action or set of related actions, a system-generated determination that a recommendation should be provided for an action or set of related actions, and/or based on other factors.

In some examples, the recommendation engine 123 may determine the set of existing actions based on an impact of each of the actions. In addition to or in lieu of a score related to the action, the recommendation may determine an impact of the action on the user. The impact may be a quantitative impact (e.g., an overall cost of health care associated with engaging in the action, a cost of health care for a predetermined time period associated with the engaging in the action, a change in life expectancy related to engaging in the action, a number of hospitalizations, and/or other quantitative impact, a qualitative impact (e.g., a change in quality of life related to engaging in the action, a likelihood the user will engage in the action, and/or other qualitative impact), and/or a combination of a qualitative and quantitative impact. In some examples, the recommendation engine 123 may determine a quantitative impact for each qualitative impact as well.

In some examples, the recommendation engine 123 may determine a quantitative impact of cost of healthcare related to the action by determining, from the multiple sets of similar healthcare decision trees, a subset of healthcare decision trees that engaged in each of the set of existing actions. The recommendation engine 123 may determine the subset of healthcare decision trees that engaged in each of the set of existing actions a predetermined time period before. For example, the recommendation engine 123 may determine the predetermined time period from a stored time period in data storage 129 (e.g., 3, years, 5 years, and/or other time frame), from a time period stored for each action available (e.g., 3 years associated with a first action, 2.5 years associated with a second action, etc.), by machine-learning or using data science to determine the appropriate time period for each action, and/or in another way to determine the time period for seeing an change in health care cost associated with engaging in the action. In some examples, the recommendation engine 123 may determine a new set of actions as the set of existing actions, based on the determined subset of healthcare decision trees, or may maintain the previously determined set of existing actions to calculate an impact.

The recommendation engine 123 may determine the impact as a cost of healthcare related to each action in the set of existing actions by determining, for each healthcare decision tree in the subset of healthcare decision trees associated with an action, an amount of money spent on healthcare since engaging in the action after the predetermined time period. The amount of money may be a total amount of money spent after the predetermined time period, an amount of money spent out of pocket after the predetermined time period, an amount of money spent by insurance after the predetermined time period, an amount of money spent directly on actions that were directly subsequent to the action, an amount of money spent on additional treatment issues that arose after engaging in the action, an amount of money spent on unexpected treatment issues that arose after engaging in the action, and/or other determination of a monetary impact of engaging in the action.

In some examples, the recommendation engine 123 may calculate the impact as a monetary impact of engaging in the action by taking an average, a sum, and/or other calculation related to each cost determined from each healthcare decision tree in the subset of healthcare decision trees. The recommendation engine 123 may store and associate the determined monetary impact as part of information stored related to the action and/or as a sub-score related to the action. [note: every impact above can be quantitatively measured, so discuss each of those impacts in a quantitative way].

The recommendation engine 123 may provide one or multiple actions in response to each action in the set of selected actions. The recommendation engine 123 may determine the one or multiple actions based on stored treatment plans, actions recommended or undertaken by users with similar healthcare decision trees, scores or other metrics related to the actions, and/or based on other factors. In addition to or in lieu of this, the recommendation engine 123 may use machine learning, decision trees, data science, statistical regressions, clustering, any of the above, and/or other mathematical or statistical models to determine the one or multiple actions in response. In addition to or in lieu of this, the recommendation engine 123 may determine the one or multiple actions based on a determined cost of a predicted health care decision tree after performing the recommended action. In addition to or in lieu of this, the recommendation engine 123 may weigh the healthcare decision trees associated with users in the second set of healthcare decision trees based on a determined weight of the social relationship between the first user and each user associated with a healthcare decision tree in the second set of healthcare decision trees, and may consider the determined weight of the social relationship when determining the set of selected actions. In addition to or in lieu of this, the recommendation engine 123 may determine the one or multiple actions based on an impact of each of the set of selected actions.

In some examples, responsive to determining the one or multiple actions, the recommendation engine 123 may determine which action to provide to the user based on prioritizing the determined one or multiple actions. The recommendation engine 123 may prioritize the one or multiple actions based on a score associated with the action, a score associated with the related actions to that action, a cost associated with each of the one or multiple actions, user preferences related to the actions, a smallest impact associated with the action, a largest impact associated with the action, an impact seen by the most associated healthcare decision trees associated with the user, any combination thereof, and/or other factors related to the one or multiple actions. In some examples, the recommendation engine 123 may determine priorities related to the one or multiple actions based on each user, such that the same action may have a different priority for a first user than a second user.

The recommendation engine 123 may provide information related to the action from the one or multiple actions with a highest priority based on the determined priorities associated with each of the one or multiple actions. The information related to the action may comprise, for example, a title related to the action, a description related to the action, a status of the action, a set of providers with performing the action, a set of treatments with performing the action, a set of users associated with performing the action, a date range within which the action is to be performed, information to be input to the system related to performing the action, information related to the impact of the action, information related to the determined priorities associated with the action, information about users in the second set of healthcare decision trees with which the first user has a social relationship that undertook a similar action, and/or other information related to the action.

In some examples, the health risk evaluation system 110 may include a vendor management 124 engine. In response to the recommendation engine 123 determining an action or set of actions to provide to the user, the vendor management engine 124 may determine, for each action to be provided, a third party vendor (e.g., a doctor, health care provider, insurer, pharmacy, fitness provider, dietician, and/or other entity associated with the health of a user) is associated with the action. In some examples, the vendor management engine 124 may determine no vendor is needed in association with performing the action. In other examples, the vendor management engine 124 may identify a single vendor associated with performing the action, or multiple sub-actions of the action that could each involve a third party vendor. For each identified vendor, the vendor management engine 124 may identify a vendor type (e.g., medical professional, insurer, pharmacy, fitness provider, and/or other entity type associated with health of a user).

Responsive to determining each vendor and vendor type associated with the action, the vendor recommendation engine 124 may provide a vendor recommendation associated for each vendor and vendor type. In some examples, data store 129 stores information related to each third party vendor associated with the health risk evaluation system 110, including, for example, vendor name, vendor id, vendor type, vendor address, vendor description, vendor capabilities, actions associated with the vendor, schedule of the vendor, ratings provided by users of the system for the vendor, feedback provided by users of the system for the vendor, pricing of the vendor, user(s) that have engaged with the vendor, third party(ies) associated with the vendor, insurance information related to the vendor, and/or other information related to the vendor. The vendor recommendation engine 124 may determine based on the action or sub-action involving a vendor, the vendor information stored by the system 110, and/or other considerations, a set of vendors to recommend to the user.

In some examples, the vendor management engine 124 may determine a recommended vendor of the determined set of vendors for each of the action/sub-actions that involve third party vendors. For example, the vendor management engine 124 may determine the recommended vendor based on a set of metrics associated with each of the set of vendors. The vendor management engine 124 may generate the set of metrics based on the results of one or multiple tests applied to each vendor. The vendor management engine 124 may determine the one or multiple tests to apply to a vendor based on the vendor type associated with the vendor.

In some examples, the vendor management engine 124 may run the one or multiple tests after providing a recommendation of the vendor to a user for the involved action/sub-action, may determine the results and store the results as metrics related to the vendor. In some examples, the vendor management engine 124 actively solicit feedback from users that have engaged with the vendor. In some of these examples, the vendor management engine 124 may determine baseline parameters and thresholds for the metrics stored, and compare the results of the vendor to the baseline parameters and thresholds. In some examples, the vendor management engine 124 may determine the baseline parameters and thresholds based on hashing, statistical measures, regressions, classifiers, clustering, Bayesian methods, machine learning, any combination thereof, and/or other mathematical calculations.

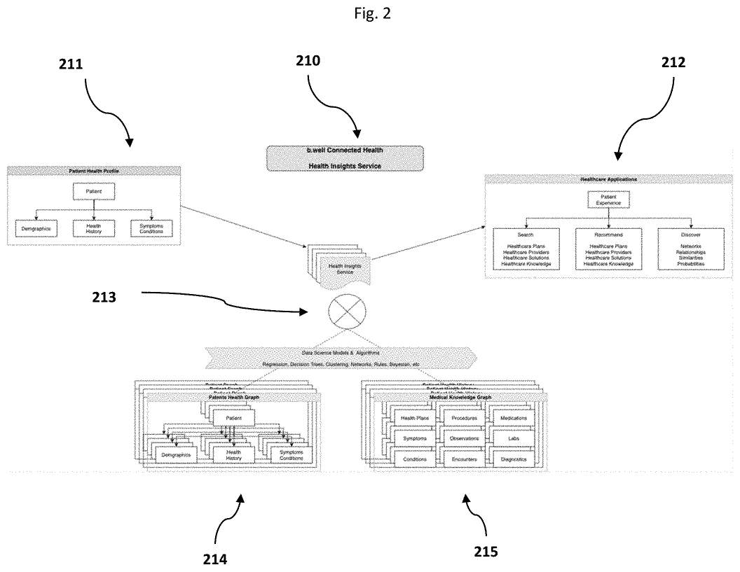

In FIG. 2, another example environment 200 is depicted in which various examples may be implemented as a health risk evaluation system 210. In the example illustrated in FIG. 2, health care profiles 211, health care applications 212, patient health graphs 214, and medical knowledge graphs are connected via a health insights service 213 which utilizes the hashing, statistical measures, regressions, classifiers, clustering, Bayesian methods, scoring, any combination thereof, and/or other mathematical calculations described herein to enable the functionality described herein. The health care profiles 211, health care applications 212, patient health graphs 214, and medical knowledge graphs 214 may represent and/or provide the data included in digital health care profiles and healthcare decision trees described above. Further, the health insights service 213 may represent the engines 121-124 described above to correlate users, and determine and provide recommendations to users related to their healthcare.

Returning to FIG. 1, in performing their respective functions, engines 121-124 may access data storage 129 and/or other suitable database(s). Data storage 129 may represent any memory accessible to health risk evaluation system 110 that can be used to store and retrieve data. Data storage 129 and/or other database may comprise random access memory (RAM), read-only memory (ROM), electrically-erasable programmable read-only memory (EEPROM), cache memory, floppy disks, hard disks, optical disks, tapes, solid state drives, flash drives, portable compact disks, and/or other storage media for storing computer-executable instructions and/or data. Health risk evaluation system 110 may access data storage 129 locally or remotely via network 50 or other networks.

Data storage 129 may include a database to organize and store data. The database may reside in a single or multiple physical device(s) and in a single or multiple physical location(s). The database may store a plurality of types of data and/or files and associated data or file description, administrative information, or any other data.

FIG. 4 is a block diagram depicting an example machine-readable storage medium 410 comprising instructions executable by a processor for dynamically evaluating health risk.

In the foregoing discussion, engines 121-124 were described as combinations of hardware and programming. Engines 121-124 may be implemented in a number of fashions. Referring to FIG. 4, the programming may be processor executable instructions 421-424 stored on a machine-readable storage medium 310 and the hardware may include a processor 411 for executing those instructions. Thus, machine-readable storage medium 410 can be said to store program instructions or code that when executed by processor 411 implements health risk evaluation system 110 of FIG. 1.

In FIG. 4, the executable program instructions in machine-readable storage medium 410 are depicted as digital health care profile instructions 421, similarity instructions 422, and recommendation instructions 423. In some examples, the executable program instructions may also include vendor management instructions 424. Instructions 421-424 represent program instructions that, when executed, cause processor 411 to implement engines 121-124, respectively.

Machine-readable storage medium 410 may be any electronic, magnetic, optical, or other physical storage device that contains or stores executable instructions. In some implementations, machine-readable storage medium 410 may be a non-transitory storage medium, where the term “non-transitory” does not encompass transitory propagating signals. Machine-readable storage medium 410 may be implemented in a single device or distributed across devices. Likewise, processor 411 may represent any number of processors capable of executing instructions stored by machine-readable storage medium 410. Processor 411 may be integrated in a single device or distributed across devices. Further, machine-readable storage medium 410 may be fully or partially integrated in the same device as processor 411, or it may be separate but accessible to that device and processor 411.

In one example, the program instructions may be part of an installation package that when installed can be executed by processor 411 to implement health risk evaluation system 110. In this case, machine-readable storage medium 410 may be a portable medium such as a floppy disk, CD, DVD, or flash drive or a memory maintained by a server from which the installation package can be downloaded and installed. In another example, the program instructions may be part of an application or applications already installed. Here, machine-readable storage medium 410 may include a hard disk, optical disk, tapes, solid state drives, RAM, ROM, EEPROM, or the like.

Processor 411 may be at least one central processing unit (CPU), microprocessor, and/or other hardware device suitable for retrieval and execution of instructions stored in machine-readable storage medium 410. Processor 411 may fetch, decode, and execute program instructions 421-424, and/or other instructions. As an alternative or in addition to retrieving and executing instructions, processor 411 may include at least one electronic circuit comprising a number of electronic components for performing the functionality of at least one of instructions 421-424, and/or other instructions.

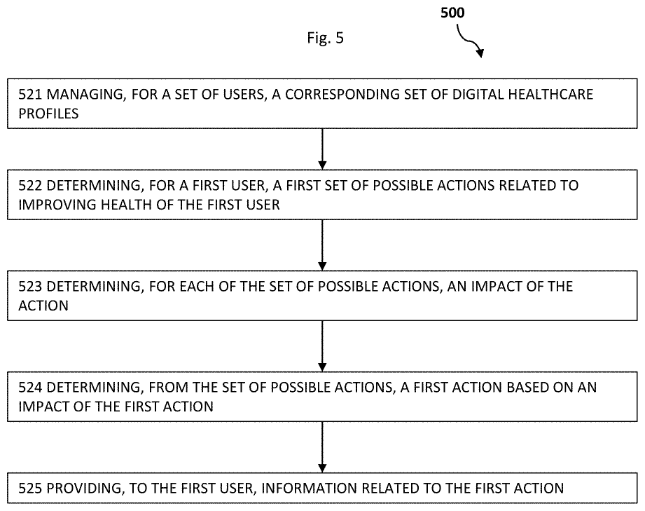

FIG. 5 is a flow diagram depicting an example method 500 for dynamically evaluating health risk. The various processing blocks and/or data flows depicted in FIG. 5 (and in the other drawing figures described herein) are described in greater detail herein. The described processing blocks may be accomplished using some or all of the system components described in detail above and, in some implementations, various processing blocks may be performed in different sequences and various processing blocks may be omitted. Additional processing blocks may be performed along with some or all of the processing blocks shown in the depicted flow diagrams. Some processing blocks may be performed simultaneously. Accordingly, method 500 as illustrated (and described in greater detail below) is meant to be an example and, as such, should not be viewed as limiting. Method 500 may be implemented in the form of executable instructions stored on a machine-readable storage medium, such as storage medium 410, and/or in the form of electronic circuitry.

In block 521, method 500 may include managing, for a set of users, a corresponding set of digital healthcare profiles, where each digital healthcare profile includes a healthcare decision tree for the corresponding user. Referring to FIG. 1, digital healthcare profile engine 121 may be responsible for implementing block 521.

In block 522, method 500 may include determining, for a first user of the set of users, a first subset of the set of users with a first set of similar digital healthcare profiles based on the corresponding set of healthcare decision trees. Referring to FIG. 1, similarity engine 122 may be responsible for implementing block 522.

In block 523, method 500 may include determining, for the first user, a second subset of the set of users that are associated with the first user based on a social relationship between the first user and each user in the second subset of user. Referring to FIG. 1, similarity engine 122 may be responsible for implementing block 523.

In block 524, method 500 may include determining, based on the first set of digital healthcare profiles of the first subset of users and the second set of digital healthcare profiles of the second subset of users, a first recommended action for the first user. Referring to FIG. 1, recommendation engine 123 may be responsible for implementing block 524.

In block 525, providing, to the first user, information related to the first recommended action. Referring to FIG. 1, recommendation engine 123 may be responsible for implementing block 525.

The foregoing disclosure describes a number of example implementations for dynamically evaluating health risk. The disclosed examples may include systems, devices, computer-readable storage media, and methods for dynamically evaluating health risk. For purposes of explanation, certain examples are described with reference to the components illustrated in FIGS. 1-5. The functionality of the illustrated components may overlap, however, and may be present in a fewer or greater number of elements and components.

Further, all or part of the functionality of illustrated elements may co-exist or be distributed among several geographically dispersed locations. Moreover, the disclosed examples may be implemented in various environments and are not limited to the illustrated examples. Further, the sequence of operations described in connection with FIG. 4 are examples and are not intended to be limiting. Additional or fewer operations or combinations of operations may be used or may vary without departing from the scope of the disclosed examples. Furthermore, implementations consistent with the disclosed examples need not perform the sequence of operations in any particular order. Thus, the present disclosure merely sets forth possible examples of implementations, and many variations and modifications may be made to the described examples. All such modifications and variations are intended to be included within the scope of this disclosure and protected by the following claims.

What is claimed is:

1. A method for dynamically evaluating health care risk, the method being implemented by machine-readable instructions, the method comprising:
managing, for a set of users, a corresponding set of digital healthcare profiles;
determining, for a first user of the set of users, a set of possible actions related to improving health of the first user;
determining, for each of the set of possible actions, an impact of the recommendation;
determining, from the set of possible recommendations, a first action based on an impact of the first action;
providing, to the first user, information related to the first action from the set of possible actions.

2. The method of claim 1, wherein determining the impact of the action comprises:
determining a money amount of the impact of the action.

3. The method of claim 2, further comprising:
determining a subset of users at a similar health stage as the first user at a predetermined period prior to the current determination;
analyzing a corresponding subset of healthcare profiles associated with the subset of users;
determining the set of possible actions based on the analysis of the corresponding subset of healthcare profiles;
determining, for each possible action, the dollar amount of the impact of the action based on a set of treatments associated with each of the set of possible actions.

4. The method of claim 1,
wherein each of the corresponding set of digital healthcare profiles comprises a corresponding healthcare decision tree, and
wherein determining the set of possible actions comprises:
determining, for a first user of the set of users, a first subset of users with a similar digital healthcare profile based on a first set of healthcare decision trees of the first subset of users; and
determining, based on correlation between a first healthcare decision tree of the first user with the healthcare decision trees of the subset of users, the set of possible actions for the first user.

5. The method of claim 4, further comprising:
determining, for the first user, a second subset of the users that are associated with the first user based on a social relationship between the first user and each user in the second subset of users;
determining, based on the first set of digital healthcare profiles associated with the first subset of users and a second set of digital healthcare profiles associated with the second subset of users, the set of possible actions for the first user.

6. The method of claim 1, wherein determining the first action comprises:
maintaining a knowledge graph related to the first set of digital healthcare profiles of the first subset of users and the second set of digital healthcare decision trees of the second subset of users; and
applying a weight to each of the digital healthcare profiles of the second subset of users based on a closeness of relationship between the first user and the corresponding user in the second subset of users.

7. The method of claim 1, further comprising:
determining that the first user executed on a first action of the set of possible actions;
updating the first healthcare decision tree based on the execution of the first action;
updating the set of possible actions based on the updated first healthcare decision tree;
determining, from the updated set of possible actions, a second action based on an impact of the second action;
providing, to the first user, the second action from the updated set of possible actions.

8. A system for dynamically evaluating health care risk, the system comprising a physical processor implementing machine readable instructions that cause the system to:
manage, for a set of users, a corresponding set of digital healthcare profiles;
determine, for a first user of the set of users, a set of possible actions related to improving health of the first user;
determine, for each of the set of possible actions, an impact of the action;
determine, from the set of possible actions, a first action based on an impact of the action;
provide, to the first user, the first action from the set of possible actions.

9. The system of claim 8, wherein determining the impact of the action comprises:
determining a money amount of the impact of the action.

10. The system of claim 8, wherein the physical processor implements machine readable instructions that cause the system to:
determine a subset of users at a similar health stage as the first user at a predetermined period prior to the current determination;
analyze a corresponding subset of healthcare profiles associated with the subset of users;
determine the set of possible actions based on the analysis of the corresponding subset of healthcare profiles;
determine, for each possible action, the dollar amount of the impact of the action based on a set of treatments associated with each of the set of possible actions.

11. The system of claim 8,
wherein each of the corresponding set of digital healthcare profiles comprises a corresponding healthcare decision tree, and
wherein determining the set of possible actions comprises:
determining, for a first user of the set of users, a first subset of users with a similar digital healthcare profile based on a first set of healthcare decision trees of the first subset of users; and
determining, based on correlation between a first healthcare decision tree of the first user with the healthcare decision trees of the subset of users, the set of possible actions for the first user.

12. The system of claim 11, wherein the physical processor implements machine readable instructions that cause the system to:
determine, for the first user, a second subset of the users that are associated with the first user based on a social relationship between the first user and each user in the second subset of users;
determine, based on the first set of digital healthcare profiles associated with the first subset of users and a second set of digital healthcare profiles associated with the second subset of users, the set of possible actions for the first user.

13. The system of claim 8, wherein determining the first action comprises:
maintaining a knowledge graph related to the first set of digital healthcare profiles of the first subset of users and the second set of digital healthcare decision trees of the second subset of users; and
applying a weight to each of the digital healthcare profiles of the second subset of users based on a closeness of relationship between the first user and the corresponding user in the second subset of users.

14. The system of claim 8, wherein the physical processor implements machine readable instructions that cause the system to:
determine that the first user executed on a first action of the set of possible actions;
update the first healthcare decision tree based on the execution of the first action;
update the set of possible actions based on the updated first healthcare decision tree;
determine, from the updated set of possible actions, a second action based on an impact of the second action;
provide, to the first user, the second action from the updated set of possible actions.

15. A non-transitory machine-readable storage medium comprising instructions executable by a physical processor of a computing device for dynamically evaluating health care risk, the machine-readable storage medium comprising:
instructions to manage, for a set of users, a corresponding set of digital healthcare profiles;
instructions to determine, for a first user of the set of users, a set of possible actions related to improving health of the first user;
instructions to determine, for each of the set of possible actions, an impact of the recommendation;
instructions to determine, from the set of possible actions, a first action based on an impact of the first action;
instructions to provide, to the first user, the first action from the set of possible actions.

16. The storage medium of claim 15, wherein determining the impact of the action comprises:
determining a money amount of the impact of the action.

17. The storage medium of claim 15, further comprising:
instructions to determine a subset of users at a similar health stage as the first user at a predetermined period prior to the current determination;
instructions to analyze a corresponding subset of healthcare profiles associated with the subset of users;
instructions to determine the set of possible actions based on the analysis of the corresponding subset of healthcare profiles;
instructions to determine, for each possible action, the dollar amount of the impact of the action based on a set of treatments associated with each of the set of possible actions.

18. The storage medium of claim 15, further comprising,
wherein each of the corresponding set of digital healthcare profiles comprises a corresponding healthcare decision tree, and
wherein determining the set of possible actions comprises:
determining, for a first user of the set of users, a first subset of users with a similar digital healthcare profile based on a first set of healthcare decision trees of the first subset of users; and
determining, based on correlation between a first healthcare decision tree of the first user with the healthcare decision trees of the subset of users, the set of possible actions for the first user.

19. The storage medium of claim 18, further comprising:
instructions to determine, for the first user, a second subset of the users that are associated with the first user based on a social relationship between the first user and each user in the second subset of users;
instructions to determine, based on the first set of digital healthcare profiles associated with the first subset of users and a second set of digital healthcare profiles associated with the second subset of users, the set of possible actions for the first user.

20. The storage medium of claim 15, further comprising:
instructions to determine that the first user executed on a first action of the set of possible actions;
instructions to update the first healthcare decision tree based on the execution of the first action;
instructions to update the set of possible actions based on the updated first healthcare decision tree;
instructions to determine, from the updated set of possible actions, a second action based on an impact of the second action;
instructions to provide, to the first user, the second action from the updated set of possible actions.
